(19) United States
(12) Patent Application Publication
(10) Pub. No.: US 2019/0085296 A1
(43) Pub. Date: Mar. 21, 2019

(54) METHOD OF EXPANSION OF HUMAN PANCREAS PROGENITOR CELLS FROM STEM CELLS USING FEEDER-CONDITIONED MEDIA

(71) Applicant: Sergio Mora, San Diego, CA (US)

(72) Inventor: Sergio Mora, San Diego, CA (US)

(21) Appl. No.: 16/136,699

(22) Filed: Sep. 20, 2018

Related U.S. Application Data
(60) Provisional application No. 62/560,950, filed on Sep. 20, 2017.

(51) Int. Cl. C12N 5/071; C12N 5/074; C12N 5/0735; C12N 5/00

(52) U.S. Cl. CPC C12N 5/0678; C12N 5/0696; C12N 5/0606; C12N 2533/52; C12N 2501/119; C12N 2501/117; C12N 2501/11; C12N 5/0037

(57) ABSTRACT

The present disclosure provides a method of producing and expanding human pancreas progenitor cells using, for example, iPSC derived cells and a human feeder cell conditioned medium. In one embodiment, cardiac mesenchyme cells are employed as feeder cells and those cells secrete growth factors, such as one or more of FGF10, KGF, or EGF, that promote pancreatic bud formation and expansion during development. In one embodiment, feeder cells are isolated from human stem cells, e.g., a human iPS-derived cardiac cells, and used to condition media and promote the growth and proliferation of iPSc derived pancreatic progenitor cells (in a feeder-free system).

### CROSS-REFERENCE TO RELATED APPLICATIONS

This application claims the benefit of the filing date of U.S. application Ser. No. 62/560,950, filed Sep. 20, 2017, the disclosure of which is incorporated by reference herein.

### BACKGROUND

Pancreatic progenitor cells are multipotent stem cells originating from the developing foregut endoderm which have the ability to differentiate into the lineage specific progenitors responsible for the developing pancreas. They give rise to both the endocrine and exocrine cells. Exocrine cells constitute the acinar cells and the ductal cells. The endocrine cells constitute the beta cells which make insulin, alpha cells which secrete glucagon, delta cells which secrete somatostatin, and the PP-cells which secrete pancreatic polypeptide.

The pancreas is an organ of endodermal origin. The endoderm is amongst the three germ layers that make up the developing embryo. The origination of the pancreatic tissue is from the dorsal and ventral aspects of the posterior foregut. They can be observed during embryonic development. Fusion of these buds occurs during rotation of the developing gut. The fused and developed pancreas includes pancreatic enzyme secreting cells (exocrine cells), digestive enzyme transporting cells (ductal cells) and hormone producing cells (endocrine cells). These endocrine cells develop in discrete areas within the pancreas known as the islets of Langerhans.

In humans, the dorsal bud can be observed 26 days post-fertilization. However, the islet cells can only be observed at 52 days post-fertilization. The development of beta cells precedes that of the development of other endocrine cells in the islets. All islet cells can be observed in the first trimester in human. This variation in the development of islet cell subtypes is due to differential gene expression and induction pathways of progenitor cells.

The endocrine precursors are a committed group of progenitors that develop into all the endocrine cells in the pancreas. Endocrine lineages develop into delta cells, PP-cells, epsilon cells, beta cells and alpha cells. Insulin, produced by the beta cells, and glucagon, produced by alpha cells, antagonistically regulate the glucose homeostasis in the mammalian body. PP-cells produce pancreatic polypeptide which is a regulator of endocrine and exocrine secretions in the pancreas and gut. Delta cells produce somatostatin which is a growth hormone inhibiting hormone and has important function in the regulation of hormone production from the anterior pituitary gland. Epsilon cells produce Ghrelin (hunger hormone) which is a. neuropeptide that acts on the hypothalamic center of the brain, where it couples with GHSR (growth hormone secretagogue receptors) and mediates hunger.

The exocrine progenitor cell develops into precursor cells expressing amylase. These cells then can be identified in tissue to be secretory in nature and contribute to the production of pancreatic enzymes.

The development of a protocol involving the directed generation of pancreatic progenitors has been performed on hESCs (human embryonic stem cells). These cells show potential in therapy for metabolic diseases of the pancreas, e.g., diabetes, and have been programmed to pancreatic progenitors using factors mimicking the developmental cues a developing endoderm would require forming functional pancreatic tissue. hESCs are grown on matrigel and then allowed to differentiate into endoderm and later defined cells under the influence of bFGF, EGF, and BMP4.

### SUMMARY

The disclosure provides for the generation and expansion of pancreas progenitor cells using, in one embodiment, human cardiac mesenchymal feeder cells derived from an induced pluripotent stem (iPS) cell line. Cardiac mesenchymal feeders secrete factors into media that favor the proliferation and expansion of human iPS-derived pancreatic progenitor cells. To enhance attachment and expansion of pancreatic progenitor cells in feeder-free conditions, tissue culture coating conditions, e.g., STMX-matrix, may be used. The combination of factors and optionally coated receptacles allows the expansion of pancreatic progenitors (eXPPs) for several passages, without altering their developmental properties. Thus, the methods herein provide for pancreatic progenitors which, in one embodiment, are obtained from induced pluripotent stem cells (iPSCs) under feeder cell-free conditions, where the pancreatic progenitors can be subsequently differentiated into endocrine and/or exocrine pancreatic cells for therapeutic, toxicology, and/or efficacy drug testing.

### BRIEF DESCRIPTION OF THE FIGURES

FIG. 1 shows the presence of a biomarker SOX17 (immunostaining for hSOX17 protein) indicative of robust definitive endoderm formation, first step for pancreatic progenitor generation. Images are captured with Image-Xpress high-throughput imaging system: 25 images per iPSC line and condition. All lines show an even expression of SOX17, with different efficiencies depending on the iPSC line. It is important to note that some lines that are refractory for differentiation (iPS-6) in GIBCO media, they show higher number of SOX17 positive cells in STMX_DE media.

FIG. 2 shows gene expression pattern by qPCR when different iPSC lines are differentiated at different time frames (stage2, posterior foregut and stage3, pancreatic progenitor).

FIG. 3 shows an image of different biomarker expression (PDX1 and PAX6 proteins) in iPS-derived pancreatic progenitors.

FIG. 4 shows an image of a typical feeder free work flow diagram of the invention.

FIG. 5 shows an image of EDTA dissociated pancreas progenitors on feeders (top panels) and in STMX matrix (bottom panels) in the presence of feeder free media.

FIG. 6 shows an image of Accutase disassociated pancreas progenitors on feeders (top panels) and in STMX matrix (bottom panels) in the presence of feeder free media.

FIG. 7 shows an image of expandable pancreas progenitors from different cell lines on STMX matrix in the presence of cardiac mesenchyme conditioned media.

### DETAILED DESCRIPTION

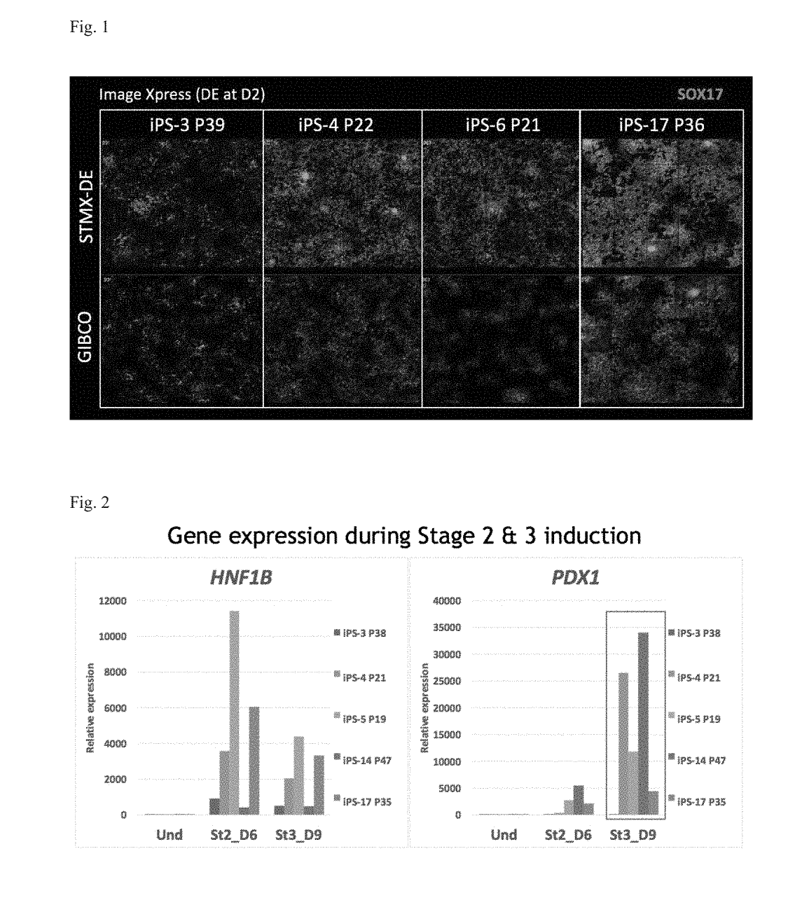

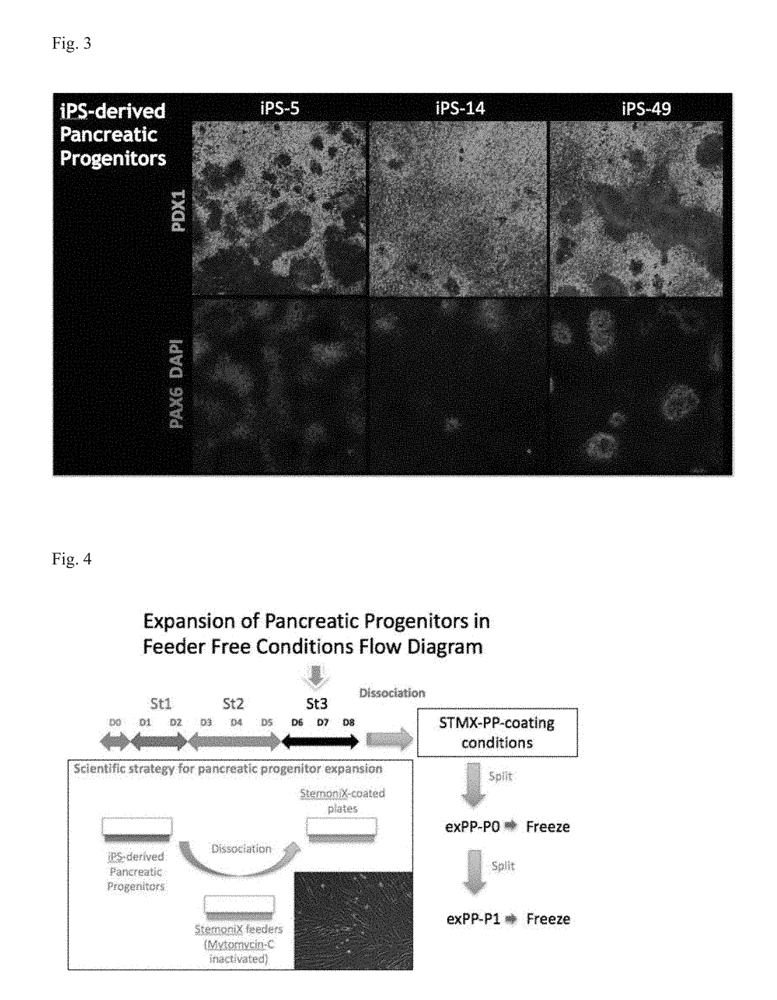

Prior to the expansion of pancreatic progenitors, an improved protocol for pancreatic differentiation, described in U.S. application Ser. No. 62/436,655, tiled on Jun. 1, 2017, the disclosure of which is incorporated by reference herein, was used along with definitive endoderm derivation in 2 days (see FIG. 2), to generate PDX1 pancreatic progenitor cells in only 8 days of differentiation. Those progenitors are subjected to dissociation and expansion according descriptions herein. However, other methods may be employed to obtain Stage 3 progenitor cells.

The first step in expansion of pancreatic progenitors is the use of a feeder culture, e.g., growing the feeder cells. Feeder cells, such as mouse cells (e.g., mitotically inactive primary mouse embryonic fibroblasts) or human fibroblasts, are used in culture protocols to inhibit stem or progenitor cells from differentiating. The feeder cells provide secreted factors, extracellular matrix, and cellular contact to inhibit stem or progenitor cells from differentiating and to maintain a normal karyotype. Generally, embryonic stein cells are plated onto feeder layers. A limitation of working with feeder cells is cell overcrowding between the feeder cells and the embryonic stem cell colonies. An additional factor in using feeder cells is to ensure that the density of the feeder cells is sufficient for the delivery of factors to maintain the cells in an undifferentiated state without depleting nutrients in the co-culture environment, which may diminish the capacity of growth of stem cell colonies.

Trott et al. (2017) demonstrated how to create expandable pancreatic progenitors using mouse feeder cells. However, to create a more human-like pancreatic progenitor culture, it is desirable to have xeno-free conditions to avoid any cross contamination or signaling from other species in a culture that is used for drug screening or regenerative medicine applications. As used herein, xeno-free means all components in a cell culture medium are derived from the same organism whether it is animal (e.g., bovine) or human. Researchers hoping to develop stem cell therapy treatments need to meet the regulatory guidelines and have a cost-effective system that is compatible with their end use. It seems clear that for therapeutic applications, the ideal cell culture medium is devoid of any animal and/or human derived components, provides equal or increased performance (e.g. cell proliferation, protein production, etc.) over serum, and is cost-effective.

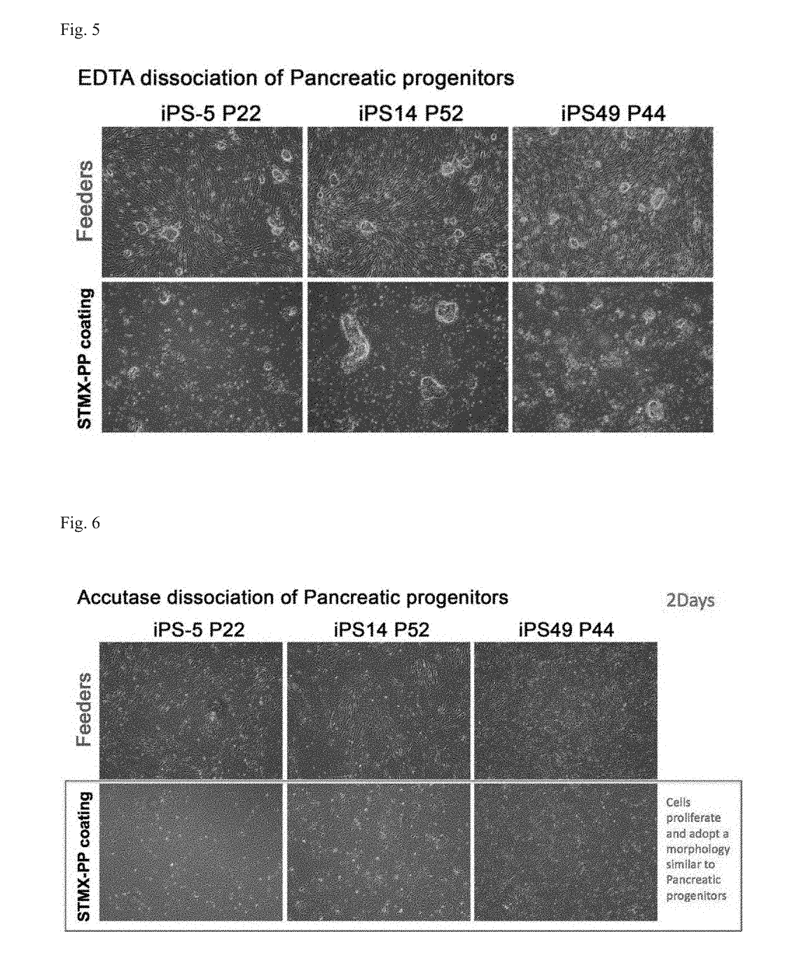

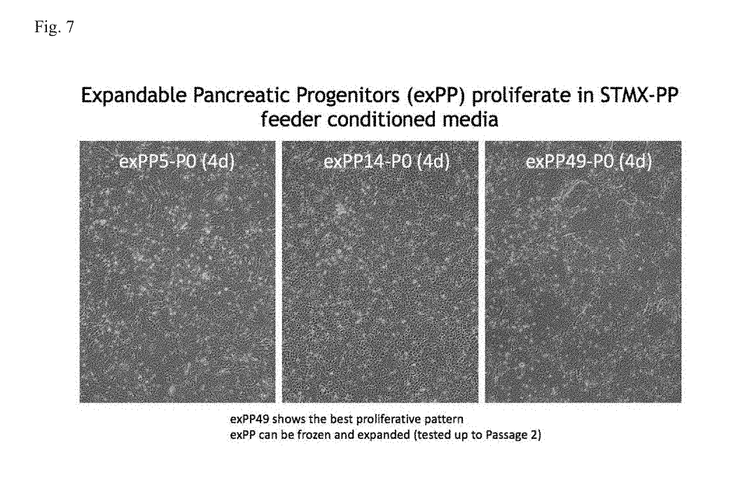

In one embodiment, a system is provided for expansion of pre-differentiated pancreatic progenitor cells from iPSCs, e.g., partially differentiated pancreatic cells. This system may employ media (e.g., STMX_PPMM; Pancreatic Progenitor Maintaining Media) that is serum-free and contains a cocktail of small molecules that modulates signaling pathways for pancreas differentiation, maturation and/or expansion. In order to mimic as much as possible the environmental and physiological cues involved in pancreas bud formation and expansion, a fibroblast-like cell line was used to provide soluble factors, which cell line was obtained after cardiac differentiation from iPSCs and selection for mesenchymal-like characteristics. For example, selection may be performed by culturing the cardiac induced differentiated cells in a media that kills myocytes and cardiomyocytes, but selects positively for other cell types (e.g., fibroblasts), or conditions that select positively for other cell types, e.g., passing cells with EDTA/trypsin, use of coated plates to culture cells, and/or shear forces. For example, after several rounds of splitting cells in gelatin-coated conditions, a homogenous layer of fibroblast-like cells is obtained. This cell line highly expresses the mesenchymal marker vimentin, giving indications of being a mesenchymal stem cell line of cardiac origin with high proliferative capacities. In one embodiment, cardiac mesenchyme secretes growth factors that promote pancreatic bud formation and expansion during development (F. C. Pan and C. Wright, 2011), e.g., cardiac mesenchyme secretes one or more of FGF10, KGF, or EGF, and/or other growth factors, cytokines, ascorbic acid, exosomes, or any combination thereof. In one embodiment a mitomycin-C inactivated cardiac mesenchyme-like cell line is employed to provide secreted, soluble factors that condition the media, e.g., STMX_PPMM. This conditioned media highly enhances the proliferation and expansion of pancreatic progenitors even in the absence of direct contact with feeder cells. FIGS. 5, 6 and 7 show cells growing in the absence of direct contact with feeders. To favor attachment and maturation of pancreatic progenitor cells, a matrix was prepared (STMX_PP_matrix) that mimics some components of pancreas extracellular matrix (ECM), e.g., see Vigier et al. and Linder et al., the disclosures of which are incorporated by reference herein. In the presence of this matrix and ROCK inhibitor (10 μM), pancreatic progenitors attach as fast as 1 hour after having been passaged. Cells are grown at high density (e.g., about 250,000 to about 500,000 cells/mL) and dissociated in clumps (e.g., using EDTA) or single cells (e.g., using Accutase) Confluency is reached quickly and cells may be subcultured every 4 to 7 days.

In one embodiment, the present disclosure allows the generation of patient specific pancreatic progenitor cells, which may be expanded and further differentiated into exocrine or endocrine cells for therapeutics, drug screening and/or basic research purposes. The lack of xenobiotics during pancreatic differentiation and expansion, makes this system highly desirable for therapeutic applications.

### Exemplary Methods and Compositions

In one embodiment, a method of expanding human pancreas progenitor cells in human feeder cell conditioned medium is provided. The method includes providing dissociated stage 3 human pancreatic progenitors and feeder cell-free conditioned media from cardiac mesenchyme-like cells; and culturing the dissociated stage 3 human pancreatic progenitors in the cell-free conditioned media that is optionally supplemented with one or more growth factors, and/or with one or more inhibitors of the TGF-β/Activin/NODAL pathway and/or with one or more inhibitors of gamma-secretase, so as to expand stage 3 pancreatic progenitors relative to stage 3 pancreatic progenitors cultured in media that is not conditioned and does not include the one or more supplemented growth factors, and/or with the one or more supplemented inhibitors of the TGF-β/Activin/NODAL pathway and/or with the one or more supplemented inhibitors of gamma-secretase. In one embodiment, the stage 3 pancreatic progenitors are derived from induced pluripotent stem cells (iPSCs). In one embodiment, the stage 3 pancreatic progenitors are derived from embryonic stem cells. In one embodiment, the dissociated stage 3 pancreatic progenitors are cultured in the conditioned media for up to 8 days. In one embodiment, the conditioned media is serum-free. In one embodiment, the cell-free conditioned media is supplemented with one or more of FGF10, KGF, or EGF. In one embodiment, the cell-free conditioned media is supplemented with one or more of dexamethasone, SB431542, or DAPT. In one embodiment, the culturing includes culturing the dissociated stage 3 pancreatic progenitors in receptacles coated with vitronectin and/or laminin. In one embodiment, the conditioned media is obtained from cardiac mesenchyme-like cells, e.g., treated with an inhibitor of DNA synthesis. In one embodiment, the dissociated cells are frozen. In one embodiment, the cells are induced to form exocrine cells. In one embodiment, the cells are induced to form endocrine cells. In one embodiment, the stage 3 human pancreatic progenitors are from a diabetic patient. In one embodiment, the cardiac mesenchyme-like cells are obtained from a population of human stem cells subjected to cardiac differentiation conditions. In one embodiment, the population comprises fibroblasts, myocytes and/or cardiomyocytes. In one embodiment, the cardiac mesenchyme-like cells comprise fibroblasts but not myocytes and cardiomyocytes. In one embodiment, the conditioned media is xenogeneic serum-free. In one embodiment, the feeder cells secrete one or more of FGF10, KGF, or EGF. In one embodiment,the feeder cells are treated with mitomycin C or irradiation. In one embodiment, the progenitor cells are cultured on extracellular matrix coated substrates. In one embodiment, the matrix comprises one or more hemopexins.

Also provided is a feeder-cell free method of expanding human pancreas progenitor cells in conditioned medium. The method includes culturing dissociated stage 3 human pancreatic progenitors in a cell-free conditioned medium obtained from human stem cells subjected to cardiac differentiation conditions which medium is optionally supplemented with one or more growth factors, and/or with one or more inhibitors of the TGF-β/Activin/NODAL pathway, and/or with one or more inhibitors of gamma-secretase, so as to expand stage 3 pancreatic progenitors relative to stage 3 pancreatic progenitors cultured in media that is not conditioned and does not include the one or more supplemented growth factors, and/or does not include the one or more supplemented inhibitors of the TGF-⊖/Activin/NODAL pathway and/or does not include the one or more supplemented inhibitors of gamma-secretase. In one embodiment, the stage 3 pancreatic progenitors are derived from induced pluripotent stem cells (iPSCs). In one embodiment, the stage 3 pancreatic progenitors are induced embryonic stem cells. In one embodiment, the dissociated stage 3 pancreatic progenitors are cultured in the conditioned media for up to 8 days. In one embodiment, the conditioned media is serum-free. In one embodiment, the cell-free conditioned media is supplemented with one or more of FGF10, KGF, or EGF. In one embodiment, the cell-free conditioned media is supplemented with one or more of dexamethasone, SB431542, or DAPT. In one embodiment, the culturing includes culturing the dissociated stage 3 pancreatic progenitors in receptacles coated with vitronectin and/or laminin, e.g., human vitronectin and/or laminin. In one embodiment, the culturing includes culturing the dissociated stage 3 pancreatic progenitors in receptacles coated with vitronectin and laminin but not collagen or fibronectin. In one embodiment, the conditioned media is obtained from cardiac mesenchyme-like cells treated with an inhibitor of DNA synthesis. In one embodiment, the dissociated cells are frozen. In one embodiment, the cells are induced to form exocrine cells. In one embodiment, the cells are induced to form endocrine cells. In one embodiment, the stage 3 human pancreatic progenitors are from a diabetic patient. In one embodiment, the cardiac mesenchyme-like cells are obtained from a population of human stem cells subjected to cardiac differentiation conditions. In one embodiment, the population of cardiac mesenchyme cells comprises fibroblasts, myocytes and/or cardiomyocytes. In one embodiment, the cardiac mesenchyme-like cells comprise fibroblasts but not myocytes and cardiomyocytes. In one embodiment, the conditioned media is xenogeneic serum-free. In one embodiment, the feeder cells secrete one or more of FGF10, KGF, or EGF. In one embodiment, the feeder cells are treated with mitomycin C or irradiation. In one embodiment, the progenitor cells are cultured on extracellular matrix coated substrates. In one embodiment, the matrix comprises one or more hemopexins.

A culture receptacle, e.g., a well in a multi-well plate, is also provided having a culture of stage 3 human pancreatic progenitors in cell-free conditioned medium, wherein the receptacle comprises vitronectin and/or laminin, and wherein the medium is optionally supplemented with one or more growth factors, and/or with one or more inhibitors of the TGF-β/Activin/NODAL pathway and/or with one or more inhibitors of gamma-secretase, wherein the medium is conditioned with medium from human stem cells subjected to cardiac differentiation.

### Exemplary Media, Reagents and Protocols:

STMX-matrix includes Vitronectin and/or Laminin 1/1 in DPBS (Dulbecco's Phosphate Buffered Saline wo Ca/Mg). Plates are incubated in STMX-matrix (e.g., 1 mL for 1 well of a 6 well plate) for at least 30 minutes at room temperature. Plates can be stored at 4° C. degrees for up to 2 weeks.

### Pancreatic Progenitors Maintaining MEDIA (PPMM)

Advanced DMEM

2 mM L-glutamine

100 U/mL penicillin/streptomycin

1× N2 supplement

1× B27 supplement

Conditioned media STMX_PPMM is obtained by maintaining STMX_PPMM for at least 24 hours on a FLC-5 line that is mitomycin-C inactivated (0.1 μg/mL, mitomycin-C in 10% FBS, DMEM high glucose, L-Glut for 24 hours). The FLC-5 line secretes factors into the STMX_PPMM media to provide for conditioned media. This conditioned media is further supplemented with some growth factors, e.g., EGF and FGF10, and small molecules, e.g., dexamethasone, retinoic acid (RA), a TGF beta inhibitor such as SB431542, and/or a gamma secretase inhibitor such as RAPT, to provide the STMX-PP expansion media. STMX-PP expansion media:

STMX_PPMM Conditioned media

30 nM dexamethasone

50 ng/mL EGF

50 ng/mL FGF10

3 μM RA

10 μM SB431542

1 μM DAPT

Other gamma secretase inhibitors useful in the media include but are not limited to indomethacin, BMS-708163, ELN-475516, RP04929097, MK0752, PF-03084014, LY-411575, LY-450139, MRK-003, GSM01, or E2012.

Other TGF-beta or activin inhibitors useful in the media include but are not limited to A 83-01, dorsomorphin, DMH-1, LDN 193189, SB505124, ML 347, LDN 212854, LDN 214117, dalantercept, fresolimumab, belagenpumatucel-L, gelunisertib, trabedersen, gemogenovatucel-T, LY2109761, pirfenidone, GW788388, LY364947, K02288, SD-208, vactosertib, or alantolactone.

### REFERENCES

Linder et al., “Immunohistochemical expression of extracellular matrix proteins and adhesion molecules in pancreatic carcinoma.,” Hepatogastroenterology., vol. 48, no. 41, pp. 1321-7, 2001.

Pan and Wright, “Pancreas organogenesis: From bud to plexus to gland,” Developmental Dynamics, vol. 240, no. 3. pp. 530-565, 2011.

Trott et al., “Long-Term Culture of Self-renewing Pancreatic Progenitors Derived from Human Pluripotent Stern Cells,” Stem Cell Reports, vol. 8, no. 6, pp. 1675-1688, Jun. 2017.

Vigier et al., “Composition and organization of the pancreatic extracellular matrix by combined methods of immunohistochemistry, proteomics and scanning electron microscopy,” Curr. Res. Transl. Med., vol. 65, no. 1, pp. 31-39, 2017.

The above discussion is meant to be illustrative of the principle and various embodiments of the present invention. Numerous variations and modifications will become apparent to those skilled in the art once the above disclosure is fully appreciated. It is intended that the following claims be interpreted to embrace all such variations and modifications.

What is claimed:

1. A method of expanding human pancreas progenitor cells in human feeder cell conditioned medium, comprising:
providing dissociated stage 3 human pancreatic progenitors;
providing feeder cell-free conditioned media from human cardiac mesenchyme-like cells; and
culturing the dissociated stage 3 human pancreatic progenitors in the cell-free conditioned media that is optionally supplemented with one or more growth factors, and/or with one or more inhibitors of the TGF-β/Activin/NODAL pathway and/or with one or more inhibitors of gamma-secretase, so as to expand stage 3 pancreatic progenitors.

2. The method of claim 1 wherein the culturing results in enhanced expansion of the stage 3 pancreatic progenitors relative to stage 3 pancreatic progenitors cultured in media that is not conditioned and does not include the one or more supplemented growth factors, and/or does not include the one or more supplemented inhibitors of the TGF-β/Activin/NODAL pathway and/or does not include the one or more supplemented inhibitors of gamma-secretase.

3. The method of claim 1 wherein the stage 3 pancreatic progenitors are induced pluripotent stem cells (iPSCs).

4. The method of claim 1 wherein the stage 3 pancreatic progenitors are induced embryonic stem cells.

5. The method of claim 1 wherein the dissociated stage 3 pancreatic progenitors are cultured in the conditioned media for up to 8 days.

6. The method of claim 1 wherein the conditioned media is serum-free, is supplemented with one or more of FGF10, KGF, or EGF, is supplemented with dexamethasone, is supplemented with SB431542, is supplemented with DAPT, or any combination thereof.

7. The method of claim 1 wherein the culturing includes culturing the dissociated stage 3 pancreatic progenitors in receptacles coated with vitronectin and/or laminin.

8. The method of claim 1 wherein the conditioned media is obtained from cardiac mesenchyme cells treated with an inhibitor of DNA synthesis.

9. The method of claim 1 wherein the cardiac mesenchyme-like cells are obtained from a population of human stem cells subjected to cardiac differentiation conditions.

10. A feeder-cell free method of expanding human pancreas progenitor cells in a conditioned medium, comprising:
culturing dissociated stage 3 human pancreatic progenitors in a cell-free conditioned medium from human stem cells subjected to cardiac differentiation conditions which medium is optionally supplemented with one or more growth factors, and/or with one or more inhibitors of the TGF-β/Activin/NODAL pathway and/or with one or more inhibitors of gamma-secretase, so as to enhance the expansion of stage 3 pancreatic progenitors relative to stage 3 pancreatic progenitors cultured in media that is not conditioned and does not include the one or more supplemented growth factors, and/or does not include the one or more supplemented inhibitors of the TGF-β/Activin/NODAL pathway and/or does not include the one or more supplemented inhibitors of gamma-secretase.

11. The method of claim 10 wherein the conditioned media is xenogeneic serum-free.

12. The method of claim 10 wherein the cardiac cells are treated with mitomycin C or radiation.

13. The method of claim 10 wherein the progenitor cells are cultured on extracellular matrix coated substrates.

14. The method of claim 13 wherein the matrix comprises one or more hemopexins.

15. The method of claim 13 wherein the matrix comprises one or more laminins.

16. The method of claim 13 wherein the matrix comprises components of human matrix.

17. The method of claim 10 wherein the expanded stage 3 pancreatic progenitors are dissociated.

18. The method of claim 10 wherein the expanded dissociated cells are frozen,

19. The method of claim 10 wherein the expanded cells are induced to form exocrine cells or endocrine cells.

20. The method of claim 10 wherein the stage 3 human pancreatic progenitors are from a diabetic patient.
